(19) United States
(12) Patent Application Publication
(10) Pub. No.: US 2007/0059264 A1
(43) Pub. Date: Mar. 15, 2007

(54) Reduction of hair growth

(76) Inventors: Gurpreet S. Ahluwalia, Wellesley, MA (US); Peter Styczynski, Wrentham, MA (US); Rajeev Kumar Passi, Marlboro, MA (US); Douglas Shander, Acton, MA (US)

Correspondence Address: FISH & RICHARDSON P.C., P.O. BOX 1022, MINNEAPOLIS, MN, 55440-1022, US

(21) Appl. No.: 11/225,939

(22) Filed: Sep. 13, 2005

(51) Int. Cl. A61K 8/28

(52) U.S. Cl. 424/66; 424/73

(57) ABSTRACT

A composition for reducing hair growth includes α-difluoromethylomithine, an antiperspirant salt or deodorant active ingredient, and a vehicle.

The invention relates to reducing hair growth in mammals, particularly for cosmetic purposes.

A main function of mammalian hair is to provide environmental protection. However, that function has largely been lost in humans, in whom hair is kept or removed from various parts of the body essentially for cosmetic reasons. For example, it is generally preferred to have hair on the scalp but not on the face.

Various procedures have been employed to remove unwanted hair, including shaving, electrolysis, depilatory creams or lotions, waxing, plucking, and therapeutic antiandrogens. These conventional procedures generally have drawbacks associated with them. Shaving, for instance, can cause nicks and cuts, and can leave a perception of an increase in the rate of hair regrowth. Shaving also can leave an undesirable stubble. Electrolysis, on the other hand, can keep a treated area free of hair for prolonged periods of time, but can be expensive, painful, and sometimes leaves scarring. Depilatory creams, though very effective, typically are not recommended for frequent use due to their high irritancy potential. Waxing and plucking can cause pain, discomfort, and poor removal of short hair. Finally, antiandrogens—which have been used to treat female hirsutism—can have unwanted side effects.

It has previously been disclosed that the rate and character of hair growth can be altered by applying to the skin inhibitors of certain enzymes. These inhibitors include inhibitors of 5-alpha reductase, ornithine decarboxylase (ODC), S-adenosylmethionine decarboxylase, gamma-glutamyl transpeptidase, and transglutaminase. See, for example, Breuer et al., U.S. Pat. No. 4,885,289; Shander, U.S. Pat. No. 4,720,489; Ahluwalia, U.S. Pat. No. 5,095,007; Ahluwalia et al., U.S. Pat. No. 5,096,911; and Shander et al., U.S. Pat. No. 5,132,293.

α-Difluoromethylomithine (DFMO) is an inhibitor of ODC. A skin preparation containing DFMO is sold under the name Vaniqa® for the treatment of unwanted facial hair growth in women. The recommended treatment regimen for Vaniqa® is twice daily. The cream base vehicle in Vaniqa® is set out in Example 1 of U.S. Pat. No. 5,648,394, which is incorporated herein by reference.

Antiperspirant and deodorant compositions are widely used personal care products. They are used primarily for controlling underarm perspiration and related malodor, respectively. Antiperspirant and deodorant compositions are available in a variety of forms, including solid sticks, soft solids, gels, roll-on liquids, creams, and aerosol and non-aerosol sprays.

### SUMMARY

In one aspect, the invention features a cosmetic composition for reducing hair growth. The composition includes DFMO and an antiperspirant salt and/or a deodorant active ingredient. In preferred embodiments, the composition further includes a hydrophilic vehicle, and the DFMO is dissolved in the vehicle. The composition can be applied to the underarm of a human and provides the combined benefits of reduced hair growth and reduced perspiration and/or reduced malodor.

In another aspect, the invention features a method of reducing hair growth by applying the aforementioned compositions to the underarm.

Other features, objects, and advantages of the invention will be apparent from the detailed description and from the claims.

### DETAILED DESCRIPTION

Preferred antiperspirant compositions include a hydrophilic vehicle, DFMO, and an antiperspirant salt. The DFMO and, in some embodiments, the antiperspirant salt are dissolved in the hydrophilic vehicle. The preferred antiperspirant composition may include, by weight, between about 10% and 70%, and more preferably between about 15% to about 60%, of the hydrophilic vehicle; between about 0.5% and 15%, and more preferably between about 1% and 10% or 1% and 5%, of the DFMO; and between about 8% and about 25% (USP) of the antiperspirant salt.

Hydrophilic vehicles include monohydric alcohols including 2-6 carbon atoms such as ethanol and isopropanol; polyhydric alcohols having 3-6 carbon atoms and 2-6 hydroxyl groups such as propylene glycol, diethylene glycol, and ethylene glycol; and water not bound to the antiperspirant salt.

DFMO, as used herein, includes DFMO and pharmaceutically acceptable salts of DFMO. DFMO exists as two stereoisomers, D-DFMO and L-DFMO. In some preferred embodiments, substantially optically pure L-DFMO is used in the composition. Substantially optically pure L-DFMO is described and defined in U.S. Pat. No. 6,743,822, which is incorporated by reference herein.

Preferred antiperspirant salts include aluminum, zirconium, and aluminum/zirconium salts.

Preferred aluminum salts are those having the general formula Al₂ (OH)₆₋ₐXₐ wherein X is Cl, Br, I or NO₃, and a is about 0.3 to about 4, preferably about 1 to 2, such that the Al to X mole ratio is about 1:1 to 2.1:1. These salts generally have some water of hydration associated with them, typically on the order of 1 to 6 moles per mole of salt. Most preferably, the aluminum salt is aluminum chlorohydrate (i.e. X is Cl) and a is about 1, such that the aluminum to chlorine mole ratio is about 1.9:1 to 2.1:1.

Preferred aluminum-zirconium salts are mixtures or complexes of the above-described aluminum salts with zirconium salts of the formula ZrO(OH)_{2-pb}Y_{b} wherein Y is Cl, Br, I, NO₃, or SO₄, b is about 0.8 to 2, and p is the valence of Y. The zirconium salts also generally have some water of hydration associated with them, typically on the order of 1 to 7 moles per mole of salt. Preferably the zirconium salt is zirconyl hydroxychloride of the formula ZrO(OH)_{2-b}Cl_{b} wherein b is about 1 to 2, preferably about 1.2 to about 1.9. The preferred aluminum-zirconium salts have an Al:Zr ratio of about 1.7 to about 12.5, most preferably about 2 to about 10, and a metal:X+Y ratio of about 0.73 to about 2.1, preferably about 0.9 to 1.5. A preferred salt is aluminum-zirconium chlorohydrate (i.e. X and Y are Cl), which has an Al:Zr ratio of about 2 to about 10 and a metal:Cl ratio of about 0.9 to about 2.1. Thus, the term aluminum-zirconium chlorohydrate is intended to include the tri-, tetra-, penta- and octa-chlorohydrate forms. The aluminum-zirconium salt complexes may also contain a neutral amino acid, preferably glycine, typically with a Gly:Zr ratio of about 1:1 to 4:1.

Examples of preferred antiperspirant salts include the aluminum and aluminum-zirconium enhanced efficacy antiperspirant salts described in U.S. Pat. No. 5,330,751 and the calcium enhanced aluminum-zirconium chlorohydrate salts described in U.S. Ser. No. 10/641,926, which is owned by the same owner as the present application and which is incorporated by reference herein, and U.S. Pat. No. 6,245,325, which also is incorporated by reference herein.

Preferred deodorant compositions include a hydrophilic vehicle, DFMO, and an antiperspirant active ingredient. The preferred composition may include, by weight, between about 10% and 95%, and more preferably between about 15% and 90%, of the vehicle; between about 0.5% and 15%, and more preferably between about 1% and 10% or 1% and 5%, of the DFMO; and between about 0.01% and about 10%, more preferably between about 0.1% and 6%, of the deodorant active ingredient.

Suitable deodorant active ingredients inhibit, suppress, mask or neutralize malodor. These may include (1) antimicrobial or bactericidal agents which kill the bacteria responsible for malodor production, (2) agents which inhibit or suppress or interfere with the bacterial enzymatic pathway that produces malodor, and (3) agents which mask or absorb or neutralize malodor. Fragrances are not considered deodorant active ingredients within the meaning of this application. Examples of deodorant actives include triclosan, triclocarban, usnic acid salts, zinc phenolsulfonate, b-chloro-D-alanine, D-cycloserine, aminooxyacetic acid, cyclodextrin, sodium bicarbonate. Antiperspirant salts may also function as deodorant actives.

The antiperspirant and deodorant compositions may also include other conventional cosmetic ingredients that provide desired properties. These ingredients include surfactants, fragrances, encapsulated fragrances, colorants, emollients, or a combination of these ingredients. Examples of emollients include stearyl alcohol, mink oil, cetyl alcohol, oleyl alcohol, isopropyl laurate, polyethylene glycol, petroleum jelly, palmitic acid, oleic acid and myristyl myristate.

The viscosity of the antiperspirant or deodorant composition may be increased if desired by including a thickening agent. Any thickening agent may be used provided it is compatible with, for example, aqueous alcoholic solutions. Particularly suitable thickening agents include the hydroxyalkyl celluloses, such as hydroxyethyl cellulose and hydroxypropyl cellulose. Other thickening agents also may be used such as carboxypolymethylene, carboxy vinyl polymer (e.g., Carbopol), sodium carboxymethyl cellulose, starches, gums, pectins, polyacrylates, etc. If included in the composition, the thickening agent will be present in an amount to provide the desired viscosity, typically about 0.01% to about 5% by weight, more typically about 0.05% to about 2%.

The antiperspirant and deodorant compositions also optionally can include components that enhance the penetration of DFMO into the skin and/or to the site of action. Examples of penetration enhancers include urea, polyoxyethylene ethers (e.g., Brij-30 and Laureth-4), 3-hydroxy-3,7,11-trimethyl-1,6,10-dodecatriene, terpenes, cis-fatty acids (e.g., oleic acid, palmitoleic acid), acetone, laurocapram, dimethylsulfoxide, 2-pyrrolidone, oleyl alcohol, glyceryl-3-stearate, propan-2-ol, myristic acid isopropyl ester, cholesterol, and propylene glycol. A penetration enhancer can be added, for example, at concentrations of 0.1% to 20% or 0.5% to 5% by weight.

When a antiperspirant or deodorant composition is in the form of liquid, a flowable composition may have, for example, a viscosity of less than 1000 cp, and preferably less than 200 cp or 50 cp. Viscosity is measured at 21° C. using a Brookfield Model viscometer with spindle # 3.

DFMO optionally can be incorporated into the antiperspirant and deodorant composition using the phase inversion procedures described in U.S. Ser. No. 10/347,987, which is owned by the same owner as the present application and is hereby incorporated by reference herein.

Examples of preferred antiperspirant compositions are provided below.

### EXAMPLE 1

| Chemical Name | Wt % |
|---|---|
| Water | 71.30 |
| Aluminum Zirconium Tetrachlorhydrex Glycine | 16.00 |
| Cyclomethicone | 7.50 |
| Steareth-2 | 2.40 |
| PPG-15 Stearyl Ether | 1.80 |
| Ceteth-20 | 0.80 |
| GPC-1519 | 0.20 |
| Total | 100.00 |

The above formulation is used in the Right Guard® Roll-On product. DFMO was dissolved in the water phase of the product at concentrations of 1% to 3%.

### EXAMPLE 2

### Al/Zr salt in a Hydroalcoholic Carrier

| Chemical Name | Wt % |
|---|---|
| Water | 49.8 |
| Aluminum/Zirconium Tetrachlorhydrex-Glycine | 18.2 |
| Ethanol | 16.0 |
| Propylene Glycol | 5.0 |
| Dipropylene Glycol | 5.0 |
| Benzyl Alcohol | 4.0 |
| Propylene carbonate | 2.0 |
| DFMO (1-3% dissolved in the water phase) | |
| Total | 100.00 |

### EXAMPLE 3

### Al Salt in a Hydroalcoholic Carrier

| Chemical Name | Wt % |
|---|---|
| Water | 55.65 |
| Aluminum chlorohydrate | 12.35 |
| Ethanol | 16.0 |
| Propylene Glycol | 5.0 |
| Dipropylene Glycol | 5.0 |
| Benzyl Alcohol | 4.0 |
| Propylene carbonate | 2.0 |
| DFMO (1-3% dissolved in the water phase) | |
| Total | 100.00 |

### EXAMPLE 4

### Al/Zr in a Nanoemulsion Carrier

| Chemical Name | Wt % |
|---|---|
| Water | 73.60 |
| Aluminum Zirconium Tetrachlorhydrex-Glycine | 18.19 |
| Glycerin | 1.60 |
| Isoceteth-20 | 2.45 |
| Glyceryl Isostearate | 1.28 |
| Dicaprylyl Ether | 2.67 |
| Germaben II (preservative) | 0.21 |
| DFMO (up to 7.5% added to the formulation) | |
| Total | 100.00 |

### EXAMPLE 5

### Al Salt in a Nanoemulsion Carrier

| Chemical Name | Wt % |
|---|---|
| Water | 76.05 |
| Aluminum chlorohydrate | 12.35 |
| Glycerin | 2.26 |
| Isoceteth-20 | 3.46 |
| Glyceryl Isostearate | 1.81 |
| Dicaprylyl Ether | 3.77 |
| Germaben II (preservative) | 0.30 |
| DFMO (up to 7.5% added to the formulation) | |
| Total | 100.00 |

The nanoemulsion AP formulation was prepared by the following procedure:

Step 1. Add water, DFMO and glycerin in a beaker and heat the mixture to 85 C.°.

Step 2. Combine Isoceteth-20, glyceryl isostearate and dicaprylyl ether in a separate beaker and heat (melt) the contents in a water bath at 85 C.°.

Step 3. Slowly add the contents of Step 1 to the mixture from Step 2, while maintaining temperature at 85 C.°. Mix for 20 minutes.

Step 4. Cool the contents to 40-45 C.° with continuous slow mixing, and then add the preservative Germall Plus to the mixture.

Step 5. Add desired AP salt (in water solution) to the step 4 mixture.

The antiperspirant and deodorant compositions should be applied topically to the armpits. The compositions may be used as an adjunct to other methods of hair removal including shaving, waxing, mechanical epilation, chemical depilation, electrolysis and laser-assisted hair removal. The composition generally is applied once or twice a day, to achieve a perceived reduction in hair growth. Perception of reduced hair growth could occur as early as 24 hours or 48 hours (for instance, between normal shaving intervals) following use or could take up to, for example, three months. Reduction in hair growth is demonstrated when, for example, the rate of hair growth is slowed, the need for removal is reduced, the subject perceives less hair on the treated site, or quantitatively, when the weight of hair removed (i.e., hair mass) is reduced.

Some of the examples described above were tested in various assays. The assay procedures will be described first, followed by the results.

Hair Mass Reduction Assay

The method employs the use of Golden Syrian hamsters. Animals were housed individually in stainless steel cages and fed a Purina Certified diet and water ad libitum. Ten week-old male hamsters were assigned to groups of 16 and hair on both sides of the back was removed with surgical clippers (No. 40 blade). Each animal was fitted with an Elizabethan collar to prevent possible ingestion of test formulations. Animals were housed in a room with a controlled environment with temperatures between 18 C.° and 26 C.° with a relative humidity of 30% to 70%. In addition, a 14/10-hour, light/dark cycle is maintained and 10 or more air changes per hour will occur.

Topical administration of test formulations occurred once per day, Monday-Friday, for a total of 13 doses per site. Ten microliters of the formulation were applied topically to each flank organ and gently spread with a pipet tip. Typically, formulations containing DFMO and an antiperspirant salt were applied to the left flank organ and the vehicle control formulation applied to the right flank organ. In situations where an accumulation of residue is noted, both flank organs on that animal (treated and vehicle control) were washed with warm water prior to the next dose. About 24 hours after the last treatment the animals were euthanized. Dorsal skin including the region around the flank organs was trimmed and final observations made. Flank organ hairs were harvested with a scalpel and weighed. Hair mass reduction was determined by calculating the mean % inhibition of hair mass for the group (typically comprised of 8 animals).

Hair Mass Reduction Assay Results

| | Hair Mass (mg) | | | |
|---|---|---|---|---|
| | DFMO | | | |
| Formulation | Dose | Composition | Vehicle Control | % Reduction |
| Example 1 | 2% | 0.48 ± .09 | 1.33 ± .23 | 65 ± 6 |
| (Al/Zr) | | | | |
| Example 2 | 2% | 1.86 ± .36 | 2.91 ± .28 | 36 ± 11 |
| (Al/Zr) | | | | |
| Example 3 | 2% | 1.30 ± .18 | 1.74 ± .27 | 11 ± 22 |
| (Al) | | | | |
| Example 2 | 2.5% | 1.77 ± .28 | 2.37 ± .29 | 26 ± 7 |
| (Al/Zr) | | | | |
| Example 3 | 2.5% | 1.31 ± .23 | 1.45 ± .18 | 32 ± .12 |
| (Al) | | | | |
| Example 2 | 3% | 1.55 ± .11 | 1.66 ± .11 | 5 ± 8 |
| (Al/Zr) | | | | |
| Example 3 | 3% | 1.15 ± .18 | 1.76 ± .28 | 32 ± 11 |
| (Al) | | | | |
| Example 4 | 3% | 0.9 ± .25 | 1.10 ± .25 | 24 ± 8 |
| (Al/Zr) | | | | |
| Example 5 | 3% | 1.19 ± .20 | 2.04 ± .19 | 41 ± 9 |
| (Al) | | | | |
| Example 4 | 7.5% | 0.87 ± .12 | 1.59 ± .20 | 38 ± 12 |
| (Al/Zr) | | | | |
| Example 5 | 7.5% | 1.32 ± .23 | 2.52 ± .25 | 43 ± 12 |
| (Al) | | | | |

Example 1, including 3% DFMO, was tested in six human subjects to determine the effectiveness of the composition for reducing hair growth. The composition was applied once a day to one armpit in a subject; Right Guard® roll-on product was applied once daily to the other armpit. The hair length measurements were made at select time intervals using a semi automated image analysis system. For these measurements, the subjects shaved both armpits and then digital images of the treatment areas (DFMO and Vehicle) were taken at 24, 48, and 72 hr later. The number of hairs measured per site ranged from 58 to 87.

The results after six weeks and 16 weeks are shown below.

Axillary Hair Growth Reduction After 6 Weeks

| | Hair Length (μm) | | |
|---|---|---|---|
| | DFMO | Vehicle Control | % Reduction |
| 24 Hrs | 274 ± 15 | 523 ± 22 | 48 ± 3 |
| 48 Hrs | 509 ± 26 | 1006 ± 40 | 49 ± 3 |
| 72 Hrs | 867 ± 34 | 1591 ± 49 | 46 ± 2 |

Axillary Hair Growth Reduction After 16 Weeks

| | Hair Length (μm) | | |
|---|---|---|---|
| | Eflornithine | Vehicle Control | % Reduction |
| 24 Hrs | 170 ± 16 | 497 ± 33 | 66 ± 3 |
| 48 Hrs | 273 ± 23 | 1002 ± 56 | 73 ± 3 |
| 72 Hrs | 326 ± 24 | 1293 ± 51 | 75 ± 2 |

Other embodiments are within the claims.

What is claimed is:

1. A composition reducing hair growth comprising α-difluoromethylomithine, dissolved in a hydrophilic vehicle, and an antiperspirant salt and/or deodorant active ingredient.

2. The composition of claim 1, wherein the composition includes from about 0.5% to about 15% of the α-difluoromethylornithine by weight.

3. The composition of claim 1, wherein the composition includes from about 1% to about 10% of the α-difluoromethylomithine by weight.

4. The composition of claim 1, wherein the composition includes from about 5% to about 80% of the hydrophilic vehicle by weight.

5. The composition of claim 1, wherein the composition includes from about 10% to about 60% of the hydrophilic vehicle by weight.

6. The composition of claim 1, wherein the hydrophilic vehicle is selected from the group consisting of water, ethanol, and propylene glycol.

7. The composition of claim 1, wherein the composition includes from about 6 USP weight percent to about 25 USP weight percent of the antiperspirant salt by weight.

8. The composition of claim 1, wherein the antiperspirant salt is an aluminum or aluminum-zirconium salt.

9. The composition of claim 1, wherein the composition includes from about 1% to about 10% of the deodorant active ingredient by weight.

10. The composition of claim 1, wherein the deodorant active ingredient is selected from the group consisting of triclosan, triclocarbon, usnic acid salt, zinc phenosulfate, b-chloro-D-alanine, D-cycloserine, aminooxyacetic acid, cyclodextrin, and sodium bicarbonate.

11. The composition of claim 1, wherein the composition includes, by weight, from about 10% to about 60% of the hydrophilic vehicle, from about 1% to about 15% of the α-difluoromethylomithine, and from about 6 USP weight percent to about 25 USP weight percent of the antiperspirant salt.

12. The composition of claim 1, wherein the composition provides a reduction in hair growth of at least 30% in the Golden Syrian Hamster assay.

13. The composition of claim 1, wherein the composition has a viscosity less than 1000 cp.

14. A method of reducing hair growth from an underarm, comprising applying to the underarm a composition including α-difluoromethylomithine, dissolved in a hydrophilic vehicle, and an antiperspirant salt and/or deodorant active ingredient.

15. The method of claim 14, wherein the composition includes from about 0.5% to about 15% of the α-difluoromethylomithine by weight.

16. The method of claim 15, wherein the composition includes, by weight, from about 10% to about 60% of the hydrophilic vehicle and from about 6 USP weight percent to about 25 USP weight percent of the antiperspirant salt by weight.

17. The method of claim 14, wherein the antiperspirant salt is an aluminum or aluminum-chlorohydrate salt.

18. The method of claim 14, wherein the α-difluoromethylomithine is incorporated into the hydrophilic vehicle using a phase inversion procedure.

19. The method of claim 14, wherein the composition is applied to the underarm using a roll-on applicator.

20. The method of claim 14, wherein the composition is applied to the underarm in the form of a stick.

21. A composition comprising α-difluoromethylomithine, an antiperspirant salt, and a vehicle.
